(19) United States
(12) Patent Application Publication
(10) Pub. No.: US 2022/0401601 A1
(43) Pub. Date: Dec. 22, 2022

(54) CONTAINER SANITIZING SYSTEM

(71) Applicant: Ravyn Williams, Burbank, CA (US)

(72) Inventor: Ravyn Williams, Burbank, CA (US)

(21) Appl. No.: 17/842,096

(22) Filed: Jun. 16, 2022

Related U.S. Application Data
(60) Provisional application No. 63/211,192, filed on Jun. 16, 2021.

(51) Int. Cl. A61L 2/10; A61L 2/26; B08B 3/02; B08B 13/00

(52) U.S. Cl. CPC A61L 2/10; A61L 2/26; B08B 3/02; B08B 13/00; A61L 2202/11; A61L 2202/122; A61L 2202/17; A61L 2202/23; A61L 2202/16; A61L 2202/14

(57) ABSTRACT

A container sanitizing system is provided. The system includes one or more containers, e.g., food containers, and a system to sanitize the containers. The container sanitizing system includes a sanitizing station adapted to receive the food containers and to irradiate the items with ultraviolet light at a sufficient intensity to sanitize and disinfect the items. The food containers are collapsible and may be folded into box-like food containers for the general storage of food, and subsequently, unfolded into a generally flat structure for sanitization within the sanitizing station. The system also includes a handheld sanitizing unit that is portable and/or mobile.

### CROSS-REFERENCE TO RELATED APPLICATIONS

This application claims priority to U.S. Provisional Application No. 63/211,192, filed on Jun. 16, 2021, the entire contents of which are hereby fully incorporated herein by reference for all purposes.

### FIELD OF THE INVENTION

This invention relates to sanitizing systems, including a food storage containers and associated ultraviolet light (UV) sanitizing stations.

### BACKGROUND

It is widely known that the world suffers from a consumer waste crisis in which products, such as single-use food containers, are used once and then disposed of into landfills. It also is widely known that for a food container to be reused, it must be fully sanitized.

Accordingly, there is a need for a food container sanitizing system adapted to sanitize food container for reuse. The presently disclosed system addresses these needs.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 shows aspects of a food container sanitizing system according to exemplary embodiments hereof;

FIGS. 2-8B show aspects of a sanitizing station according to exemplary embodiments hereof;

FIGS. 9A-11B show aspects of a food container according to exemplary embodiments hereof;

FIGS. 12-13 show aspects of a handheld food container sanitizing unit according to exemplary embodiments hereof; and

FIG. 14 shows bottom, side, and top views of a handheld food container sanitizing unit according to exemplary embodiments hereof.

### DETAILED DESCRIPTION OF EXEMPLARY EMBODIMENTS

In general, and according to exemplary embodiments hereof, a container sanitizing system is provided. In some embodiments, the system includes one or more containers and a system to sanitize such containers.

As described herein, the container sanitizing system includes a sanitizing station adapted to receive items (e.g., food containers) and to irradiate the items with ultraviolet light at a sufficient intensity to sanitize and disinfect the items. The container sanitizing system also may include one or more collapsible food containers adapted to be folded into box-like food containers for the general storage of food, and subsequently, unfolded into a generally flat structure for sanitization within the sanitizing station. Other types of food containers and/or food-related items also may be provided. The system also includes a handheld sanitizing unit that is portable and/or mobile.

For the purposes of this specification, the sanitizing system 10 will be described primarily with respect to its use with sanitizing food containers. However, it is understood that the system 10 may be used to sanitize any type of item that may benefit from its functionalities and that the scope of the system 10 is not limited in any way by the type of item that it may be used with.

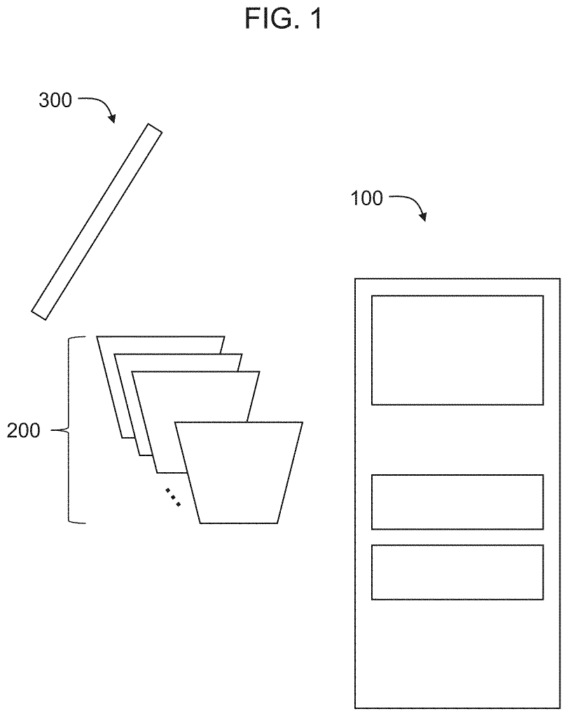

In some embodiments as shown in FIG. 1, the sanitizing system 10 (also referred to herein as simply the system 10) includes a sanitizing station 100, one or more types of food container assemblies 200, and a handheld sanitizing unit 300. In general, the sanitizing station 100 provides sanitizing services to food containers 202 (and/or other items) as a standalone unit, the food container assembly 200 includes the one or more food containers 202 adapted for use with the system 10, and the handheld sanitizing unit 300 provides sanitizing services to the food containers 202 (or other items) as a portable unit. In this way, the food containers 202 may be used to carry and/or eat food, and subsequently, be sanitized by the sanitizing station 100 and/or using the handheld sanitizing unit 300. The sanitized containers 202 may then be reused. The sanitizing system 10 also may include other elements and/or components as necessary to perform its functionalities.

Sanitizing Station 100

In some embodiments, the system 10 includes a sanitizing station 100 adapted to sanitize one or more food container assemblies 200 (e.g., food containers 202, foldable containers 204, non-foldable containers 206, food-related utensils 208, etc. as described in other sections).

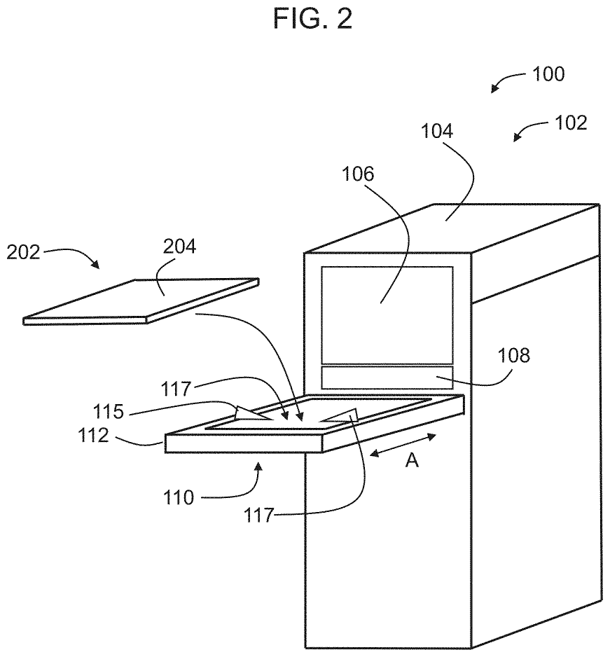

In some embodiments as shown in FIG. 2, the sanitizing station 100 includes an ultraviolet germicidal irradiation (UVGI) system 102 configured to sterilize one or more food container assemblies 200 using ultraviolet (UV) light. In some embodiments, the UVGI station 102 may include a housing 104, a display 106, a control pad 108, and a container input mechanism 110. In some embodiments, the display 106 may include a touchscreen display 106 and may provide the functionality of the control pad 108. The display 106 also may provide information regarding the use of the system 10, advertising, and other functionalities.

The container input mechanism 110 is adapted to receive a food container assembly 200 (and/or other item(s)) and input it into a cleaning chamber 120 within the inner volume of the housing 104. Once within the cleaning chamber 120, the food container assembly 200 may be sanitized by the station 102.

In some embodiments, the cleaning chamber 120 may include at least some of the following:
(i) a UVGI only cleaning chamber 122 wherein the item is cleaned using UVGI only;
(ii) a UVGI+water cleaning chamber 124 wherein the item is cleaned using UVGI and water, with the water preferably being delivered under pressure (e.g., via one or more water jets), heated, etc. The water jets may be positioned anywhere within the chamber 120 with respect to the item to be cleaned (e.g., below, to the sides, above, etc.), may be moveable (automatically, or manually), e.g., along tracks or rails, may rotate (via ball joints, automatically or manually), etc.
(iii) a UVGI+steam cleaning chamber 126 wherein the item is cleaned using UVGI and steam (e.g., steam under pressure via water steam nozzles);
(iv) a UVGI+cleaning solution cleaning chamber 128 wherein the item is cleaned using UVGI and a cleaning solution (e.g., a foam or liquid cleaning solution under pressure). In this case, the item also may require rinsing (e.g., with water) after the application of the cleaning solution;
(v) and/or any combinations thereof.

In some embodiments, the cleaning chambers 124, 126, 128 may include an option to turn on/off the UVGA functionality and/or the water, steam, cleaning solution functionalities, respectively, depending on the type of cleaning required. In some embodiments, the cleaning chambers 124, 126, 128 may first apply the water, steam, cleaning solution, respectively, and then the UVGA sanitation, however, it is understood that these operations may be implemented in other orders. In some embodiments, the cleaning chamber 120 also may include blower vents 148 (explained in other sections) that may output air (e.g., heated air under pressure) that may assist in the cleaning and/or drying of the items.

In some embodiments as shown in FIG. 2, the container input mechanism 110 includes a tray 112 that may extend out from and be retracted into the housing 104 as shown by arrow A. As such, the food container assembly 200 (a food container 202, and preferably, a collapsible food container 204 in its collapsed and generally flat state) may be placed onto the tray 112 and fed into the station 102. The tray 112 may be controlled via the control pad 108 (and/or a touchscreen display 106). In use, a user may control the station 102 to extend the tray 112 and/or this may be performed automatically by the station 102. The user may then place the food container 202 onto the tray 112, and subsequently control the tray to retract the tray 112 thereby inputting the tray 112 and the container 202 into the station's sanitizing chamber 120. Once the station 102 has sanitized the container 202, the station 102 may automatically self-extend the tray 112 for the removal of the sanitized container 204 or may be controlled by the user to do so.

In some embodiments, the tray 112 includes a recess 115 or cavity adapted to receive a collapsible food container 204 in its unfolded and generally flat state. For example, the recess 115 may be about 1″ deep, and preferably about 0.25″-0.5″ deep. In addition, the tray 112 may include securing mechanisms 117, such as clips, tabs, bands, a transparent lid that may be folded down over the top of the recess (or at least over a portion thereof), or other types of securing mechanisms that may hold the food container 204 generally flat upon the tray (and preferably within the recess 115) during the ensuing sanitation process. In other embodiments, the clips, tabs, or other securing mechanisms may be configured within the housing 104.

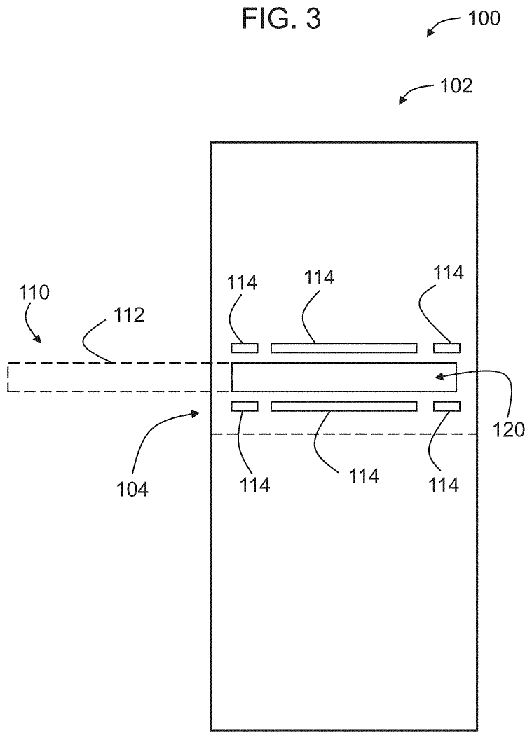

In some embodiments as shown in FIG. 3, the UVGI station 102 includes one or more irradiators 114 (e.g., UV irradiators) within its housing 104 positioned above, below, to the side(s) and/or in any position(s) relative to the tray 112 and/or to the container 202 held by the tray 112 to adequately sterilize the food container 202 as required. Any number of irradiators 114 may be used.

In some embodiments, one or more irradiators 114 may be stationary, while in other embodiments, one or more irradiators 114 may be moveable, either automatically by the system 10 and/or controlled manually (e.g., on tracks, rails, wheels, movable mounts, ball joints, etc.). When moveable, the irradiators 114 may move in any direction (e.g., laterally, up, down, diagonally, along any path as required, etc.) and/or in any orientation (e.g., may swivel, rotate, etc.). In this way, the irradiators 114 may be moved to any positions and/or orientations to ensure that each portion of the food container 202 is adequately irradiated with sufficient UV light in order to adequately sanitize the container 202.

In some embodiments, the chamber 120 may include one or more mirrors placed adjacent to the tray 112 and/or the food container 202 in any orientation so that UV light may reflect within the chamber 120 to irradiate the container 202 from multiple directions simultaneously.

It is understood that the irradiators 114 emit UV light at an adequate intensity to adequately sanitize each portion of the food container 202. In some embodiments, the irradiation process may take about 3-5 seconds, however, the irradiation process may take any amount of time, as necessary.

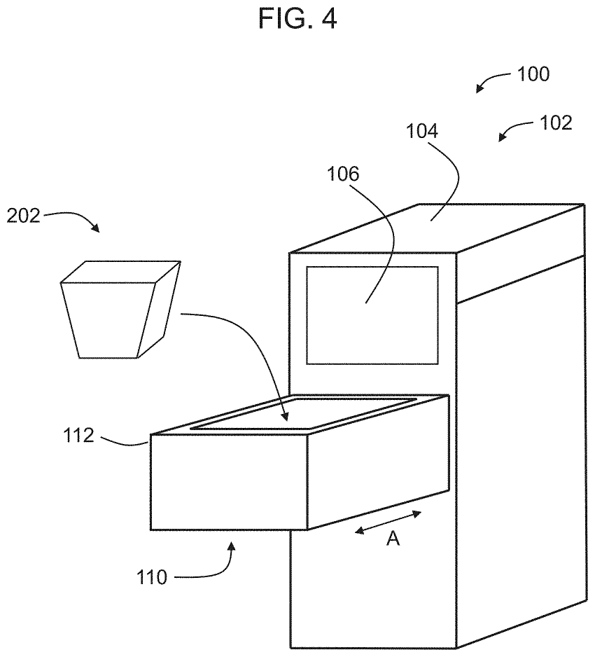

In some embodiments as shown in FIG. 4, the container input mechanism 110 may include a larger container input mechanism 110, e.g., a larger tray 112 or drawer of sufficient size to receive more than one non-collapsible food containers 206 (as described in other sections) and/or other types of larger items.

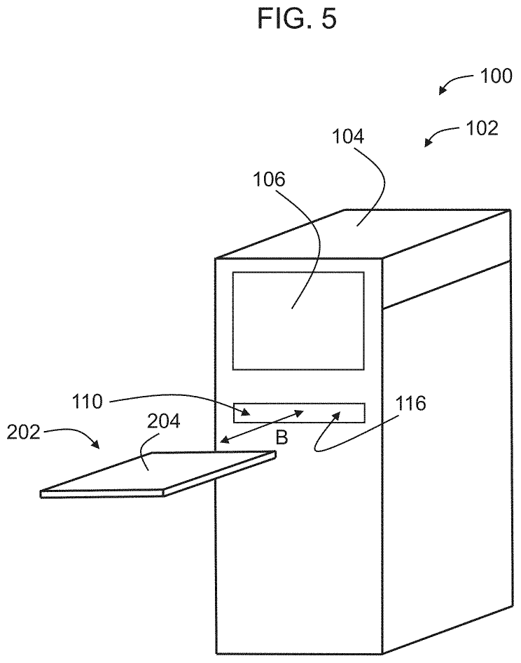

In some embodiments as shown in FIG. 5, the input mechanism 110 includes a feeding mechanism (e.g., a roller feeder) into which an unfolded and generally flat foldable container 204 may be inserted and fed into the station 102. Once inside, the foldable container 204 may be sanitized by the irradiators 114 as described above with respect to FIG. 3.

In some embodiments, the input mechanism 110 includes a conveyer belt such that items to be cleaned may be placed on the conveyer belt and thereby automatically moved into the station's internal cleaning chamber 120. In some embodiments, the conveyer belt is transparent so that UV light emitted by the irradiators 114 may pass through the belt and irradiate the item. In other embodiments, the item may be reoriented while inside the chamber 120 (e.g., lifted, rotated, etc.) in order to be fully irradiated. Once cleaned, the conveyer belt may remove the item from the chamber 120, e.g., out the same direction as input by reversing the conveyer, out a different direction (e.g., out the back), or in any other way.

In any of the embodiments described herein or otherwise, the station 102 may receive a single food container 202 at a time for sanitization, and/or may receive multiple food containers 202 simultaneously and/or sequentially. For example, the station 102 may include multiple container input mechanisms 110, e.g., multiple trays 112, multiple feeders 116, and/or any combinations thereof, capable of receiving multiple containers 202 simultaneously. In some embodiments, the container input mechanisms 110 may include a plurality of slots that may each receive a container 202 so that a plurality of containers 202 may be input into the station 102 simultaneously.

In some embodiments, the system 10 includes a food container return station 118 into which customers may return used food containers 202 without necessarily first cleaning or sanitizing the containers 202. For example, restaurant cafeterias (such as those found at universities, hospitals, large corporations, etc.) may offer food items served in food containers 202. Then, after a customer may finish with the food item and the associated food container 202, the customer may place the used food container 202 into the food container return station 118. Accordingly, it is preferable that the food container return station 118 be capable of containing multiple used food containers 202 at one time. The facility may then collect the used food containers 202 from the food container return station 118 and wash them using a sanitizing station 102, a handheld sanitizing unit 300, other types of washing methods, and/or any combinations thereof.

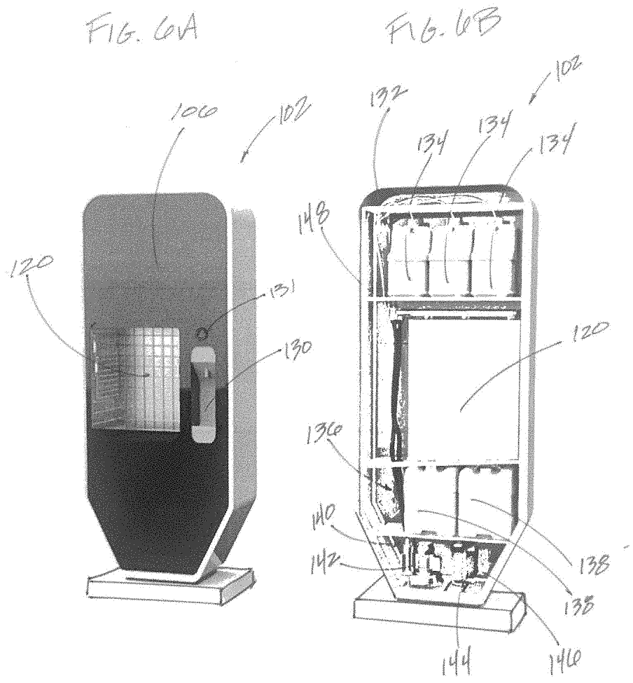

FIGS. 6A-6B, 7A-7B, and 8A-8B show additional embodiments of sanitizing stations 102 according to exemplary embodiments hereof.

In some embodiments, as shown in FIGS. 6A and 6B, the system 10 includes a sanitizing station 102 including a cleaning chamber 120 accessible from its front side, a front-side display 106 arranged above the cleaning chamber 120, and a hand sanitizer 130.

In some embodiments, the cleaning chamber 120 incudes (i) a UVGI only cleaning chamber 122, (ii) a UVGI+water cleaning chamber 124, (iii) a UVGI+steam cleaning chamber 126, (iv) a UVGI+cleaning solution cleaning chamber 128, and/or any combinations thereof. In some embodiments, the cleaning chamber 120 may include options to turn on/off the UVGA functionality, the water functionality, the steam functionality, the cleaning solution functionality, and/or any combinations thereof simultaneously and/or sequentially (in any order), depending on the type of cleaning required.

In some embodiments, the cleaning chamber 120 includes a front door that opens to allow for items to be placed into the chamber 120 for cleaning and that closes during the cleaning process. During the cleaning process, the door may be locked so that it may not be inadvertently opened. The door may be automatic, manual, and/or any combinations thereof. When the door is open, non-UV lights may illuminate the area within the chamber 120 to help the user to insert and properly arrange the items to be cleaned. The door also may be configured with a sensor that determines when an item is placed incorrectly within the chamber (e.g., too close to the front opening) thereby disallowing the door to close until the item's placement is corrected.

In some embodiments, as shown in FIG. 6A, the cleaning chamber 120 may include an internal cage 121, e.g., comprising metal or plastic grids, designed to hold the items being cleaned in the proper area within the chamber 120 and a proper distance from the UV irradiators 114. The internal cage 121 also may include fold-down shelves, slots, and/or other types of surfaces upon which items to be cleaned may be placed. The racks also may be configurable into one or more shelves to hold multiple items, etc. The racks also may be removable, interchangeable, and/or replaceable.

In some embodiments, as shown in FIG. 6B, the cleaning chamber 120 includes a bottom surface that is slanted, e.g., from left to right, or vice versa, so that water and/or debris removed from the items being cleaned may be guided down and to the right (or left) within the chamber 120 (by gravity, running water, etc.). The lower portion of the chamber's bottom surface may include an outlet for the water and/or debris so that the water and debris may be removed (preferably automatically) and stored within a collection tank below. This will be described in detail in other sections.

In some embodiments, as shown in FIG. 6A, the hand sanitizer 130 is accessible from the front of the sanitizing station 102, although it is contemplated that the station 102 may include one or more hand sanitizers 130 accessible from any side(s) of the station 102. In some embodiments, the hand sanitizing station 130 includes a chamber into which a user may place his/her hands to receive sanitizing solution (e.g., sanitizing foam disinfectant and/or gel). The dispensing of the sanitizing solution may be triggered by a sensor 131 (e.g., a camera, a motion sensor, heat sensor, etc.) configured with the station 130. In other embodiments, the dispenser may be manual.

In some embodiments, the sensor 131 also may recognize the presence of a person in front of the sanitizing station 102 and may activate its display 106 to greet the person, to instruct the person how to use the station 102, may guide the person through the process of using the station 102, may provide advertising to the person, may otherwise interact with the person, and/or any combinations thereof. In some embodiments, the sensor 131 may be designed to read a code presented by the user (e.g., a QR code) to begin the cleaning process.

In some embodiments, the sanitizing station 102 includes an internal volume partitioned into a plurality of individual areas and/or compartments. For example, in some embodiments, as shown in FIG. 6B, the station 102 may include a first internal compartment 132 (e.g., above the cleaning chamber 120) designed to store one or more first containers 134, a second internal compartment 136 (e.g., directly below the sanitizing chamber 120) designed to store one or more second containers 138, a third internal compartment 140 designed to store additional equipment (e.g., a blower 142, a debris catcher 144, an evaporator 146, and other equipment), other internal compartments, and any combinations thereof. The debris catcher 144 may collect and store debris removed from the items being cleaned, and the evaporator 146 may collect water from the cleaning chamber 120 and cause the water to be evaporated for removal.

In some embodiments, the first containers 134 include containers of sanitizing solution designed to provide sanitizing solution to cleaning chambers 120 that include UVGI+cleaning solution cleaning chambers 128. In some embodiments, the second containers 138 include containers of water designed to provide water to cleaning chambers 120 that include UVGI+water cleaning chambers 124. It is understood that the first and second containers 134, 138 may be located in any chambers 132, 136, 140 and may contain any types of materials as needed by the system 10.

In some embodiments, as shown in FIG. 6B, the cleaning station 102 includes one or more blower vents 148 in fluid communication with the blower 142 and designed to blow air (heated, cold, room temperature, etc.) from the blower 142 into the cleaning chamber 120. In this way, items within the chamber 120 that become wet or damp during the cleaning process may be dried by the incoming air streams from the vents 148.

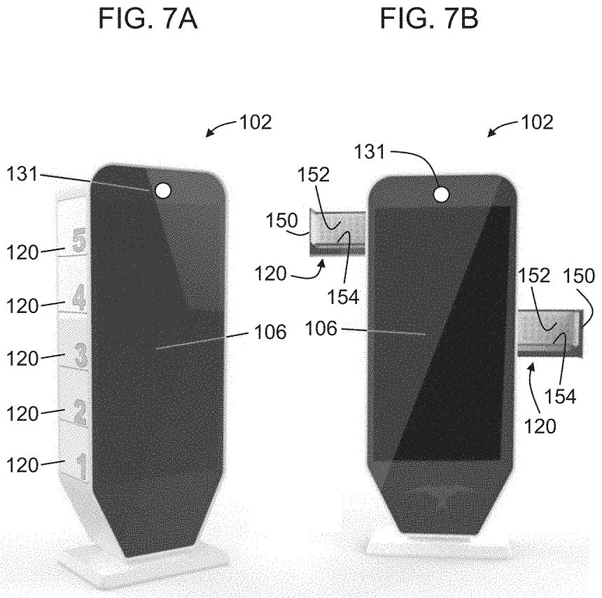

In some embodiments, as shown in FIGS. 7A and 7B, the system 10 includes a sanitizing station 102 including a front-side display 106 and a plurality of container input mechanisms 110 accessible on the left and/or right sides of the station 102. For example, as shown in FIG. 7A, the station 102 includes five container input mechanisms 110 in the form of drawers 113. FIG. 7A shows the drawers 113 closed and FIG. 7B shows two of the drawers 113 open to receive items to be cleaned.

In some embodiments, each drawer 113 leads to a dedicated cleaning chamber 120, while in other embodiments, two or more of the drawers 113 may lead to the same cleaning chamber 120.

In some embodiments, as shown in FIG. 7B, each drawer 113 includes an outer sidewall 150, a back sidewall 152, and a bottom 154. Notably, the drawers 113 do not include front sides or top sides. In this way, when the drawer 113 is open, a user may insert the item to be cleaned into the drawer 113 front the front side and/or the top (e.g., while standing in front of or to the side of the station 102). However, because the drawer 113 includes a back sidewall, a user standing at the backside of the station 102 may not access the open drawers 113.

In some embodiments, the sanitizing station 102 is operable from the front side and from the back side simultaneously. That is, a first person may interact with the station 102 via a first display 106 on the station's front side and a second person may interact with the station 102 via a second display 106 on the station's back side. In this case, a first set of drawers 113 may be oriented to serve the first person standing at the front side as described above with reference to FIG. 7B while a second set of drawers 113 may be oriented to serve the second person standing at the station's back side. The front serving drawers 113 may include open front sides facing forward and closed back sidewalls 152 in the back so that the person at the back of the station 102 may not have access to the front serving drawers 113. The back serving drawers 113 may include open fronts facing rearward and back sidewalls 152 facing the front of the station 102 so that the person at the front of the station 102 may not have access to the back serving drawers 113.

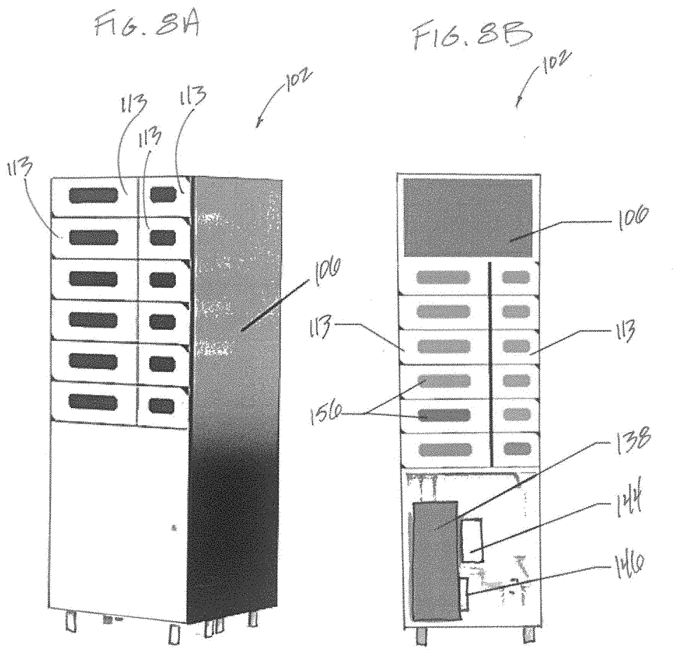

In some embodiments, as shown in FIGS. 8A and 8B, the sanitizing station 102 includes drawers 113 accessible from the front of the station 102 and a side panel display 106. Notably, some of the drawers 113 may be larger than others, e.g., some drawers 113 may be designed to hold multiple items and/or larger items while other drawers 113 may be designed to hold single and/or smaller items.

In some embodiments, the face of the drawers 113 include indicator lights 156 that indicate when a particular drawer 113 is in use or is free to use. For example, an indicator light 156 may shine green light when the drawer 113 is available for use and may shine red light when the drawer 113 is currently in use or otherwise unavailable.

In some embodiments, the sanitizing stations 102 may be designed to be mounted to a wall, on the underside of a table, on the top of a table, on the ground, hanging from the ceiling, in other areas, and/or in any combinations thereof.

### Food Container Assembly 200

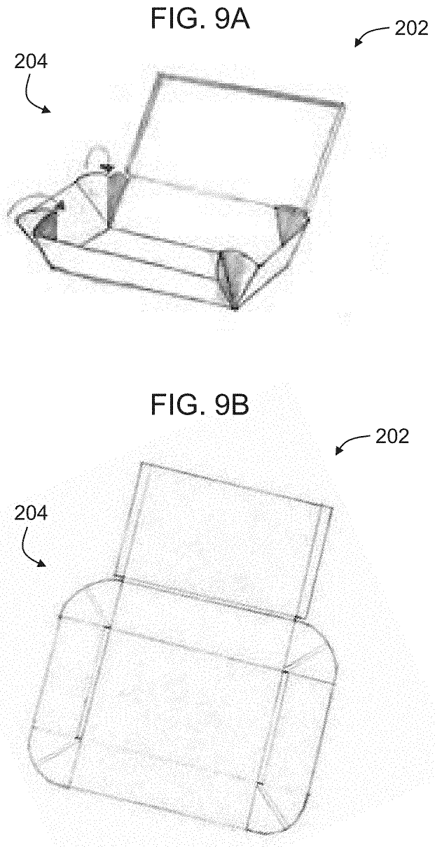

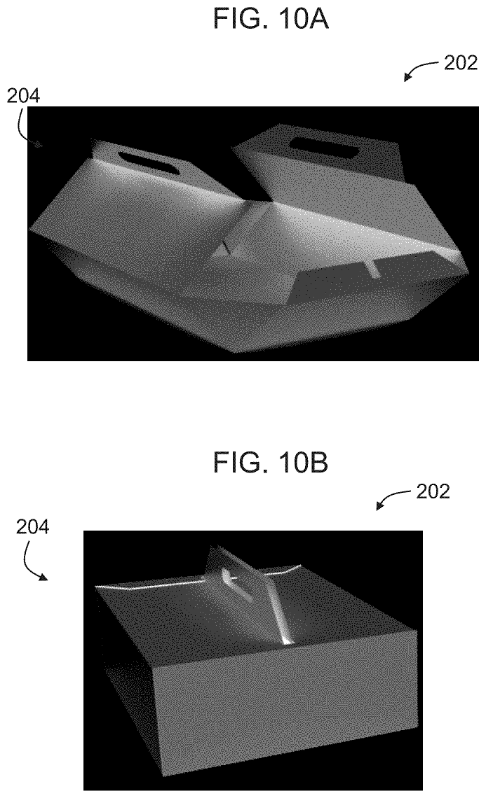

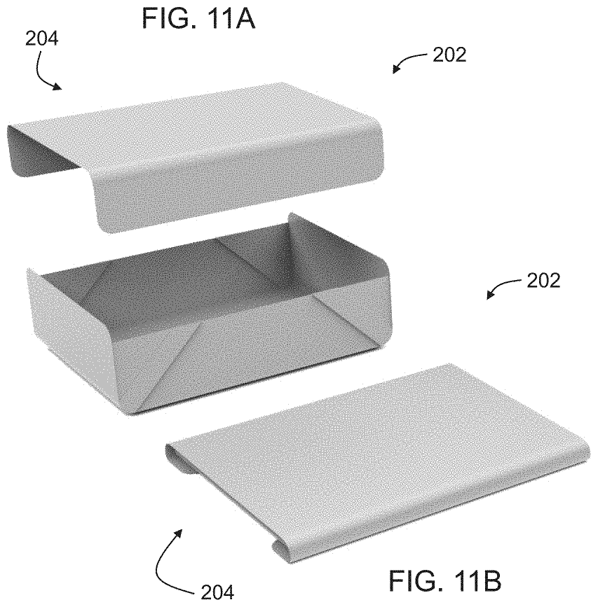

In some embodiments the food container assembly 200 includes one or more food containers 202. In some embodiments as shown in FIGS. 9A-9B, 10A-10B, and 11A-11B, the food containers 202 may include collapsible and/or foldable containers 204 that may be formed into a food container 202 with an area to store or otherwise contain the food (e.g., an inner volume, an upper surface, etc.) for use (see FIG. 9A), and that may subsequently be collapsed (unfolded) into a generally flat structure for use with the sanitizing station 100 (see FIG. 9B). In this way, the collapsed container 204 may be easily inserted into and sanitized by the sanitizing station 100 as will be described in other sections.

In some embodiments, the dimensions of the foldable container 204, when in its collapsed, unfolded and generally flat state, may range from about 2″×2″ (for small food items, condiments, etc.) to about 24″×24″ (for larger sized food items), or any combinations of these dimensions thereof. For example, in some embodiments, the foldable container 204 may include a small size with dimensions of about 9″×12″ and a larger size with dimensions of about 12″×24″. However, it is understood that the foldable container 204 may include any suitable dimensions and that the scope of the foldable container 204 is not limited in any way by its dimensions. It is contemplated that in its unfolded state, the foldable container 204 may be stored in one's purse, pocket, backpack, fanny pack, etc. for use at any food serving facility.

While the foldable containers 204 is depicted in FIGS. 9A-9B, 10A-10B, and 11A-11B as cuboid box-like containers, the foldable food container(s) 204 may be formed as any suitable shape and/or form. For example, the foldable container 204 may include a shape of any suitable prism. In other embodiments, the foldable container 204 may include a cylindrical shape. It is understood that any food container 202 (foldable or not) may include any suitable shape or form and that the scope of the food container assembly 200 and of the system 10 is not limited in any way by the shape or form of the containers 202 that it may use and support.

In other embodiments, the food container(s) 202 may not necessarily be collapsible (e.g., not foldable) and may include any type(s) of non-collapsible food containers 206 such as, without limitation, plates, bowls, cups, trays, bottles, cans, saucers, glasses, water bottles, pans, pots, boxes, jars, other types of food containers 202, and any combinations thereof. It is understood however that these containers also may include some degree of collapsibility, e.g., a cup may include a twist-off cap and a collapsible body.

In some embodiments, the food container assembly 200 also includes food-related utensils 208 such as, without limitation, forks, knives, spoons, serving utensils, ladles, sieves, spatulas, tongs, stirrers, straws, other types of utensils 208, and any combinations thereof.

In some embodiments, the food containers 202, the collapsible food containers 204, the non-collapsible food containers 206, and the food-related utensils 208 may comprise plastic, paper-based materials, glass, metal, wood, composite materials, other suitable materials, and any combinations thereof.

In some embodiments, the food containers 202, the collapsible food containers 204, the non-collapsible food containers 206, and the food-related utensils 208 comprise materials that are recyclable, eco-friendly, re-usable, microwave-safe, freezer-safe, and dishwasher-safe.

Handheld Sanitizing Unit 300

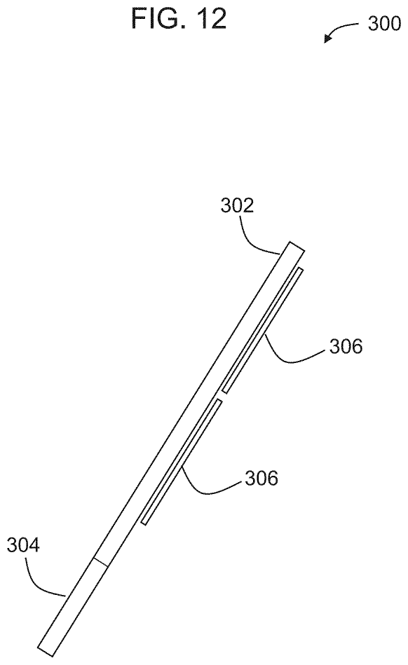

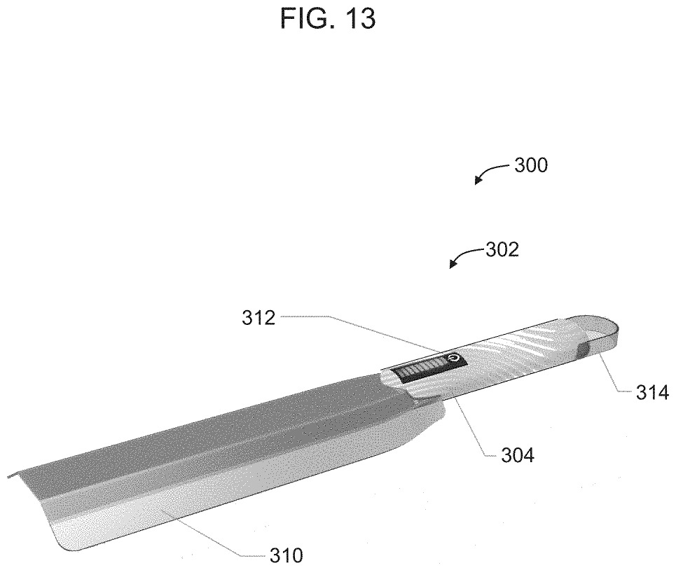

In some embodiments, as shown in FIG. 12, the system 10 includes a handheld sanitizing unit 300 (also referred to as a sanitizing wand). The handheld sanitizing unit 300 may include a body 302 (e.g., an elongate body) with a handle 304 and one or more UV irradiators 306 adapted to emit UV light at an adequate intensity to adequately sanitize at least a portion of an item e.g., a food container assembly 200. The unit 300 may be powered using rechargeable power sources (e.g., rechargeable batteries), may be coupled to a sanitizing station 102 to receive power, may include a power jack that may be plugged into an electrical outlet, may use other types of power sources, and any combinations thereof.

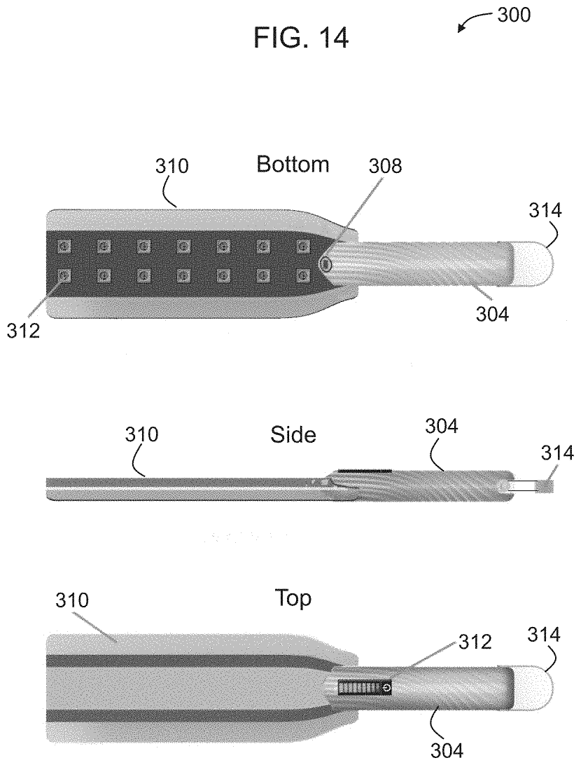

In some embodiments, as shown in FIG. 14, the unit 300 includes a motion detector 308 to sense when the unit 300 is moved. For example, if the unit is turned on (the UV irradiators 306 are radiating UV light) and the unit 300 is placed face down on a tabletop (so that the irradiation is into the top of the table), and the unit 300 is subsequently picked up by a user, the motion detector 308 will detect the movement and turn the UV irradiators 306 off. In this way, the irradiators 306 will not inadvertently irradiate UV light in a dangerous direction (e.g., towards the user's eyes). In some embodiments, it may be preferable for the motion detector 308 to be located on the underside of the unit 300 close to the irradiators 306 so that motion of the irradiators 306 may be sensed immediately upon any slight movement thereby deactivating the irradiators 306 as quickly as possible.

In some embodiments, the unit 300 also may include a UV light filtering shield 310 extending over the top of the irradiators 306 so that UV light emanating from the irradiators 306 may not extend upward towards the user. That is, any UV light directed upwards will be filtered by the UV shield 310.

In some embodiments, the unit 300 also may include a power indicator 312 and a handle loop 314 for hanging the unit on a hanger (e.g., on a wall hanger).

In use, a user may hold the handheld sanitizing unit 300 by its handle 304 such that its irradiators 306 emit UV light onto a food container assembly 200 in order to sanitize said assembly 200.

In some embodiments, the handheld unit 300 is adapted for use with a sanitizing station 102 while in other embodiments the unit is 300 adapted to be used on its own.

In some embodiments, a sanitizing station 102 may include a docking station of a handheld unit 300 for stowage and charging. In some embodiments, the system 10 also includes a tote bag or other type(s) of carrying bags for carrying one or more food container assemblies 200, one or more handheld sanitizing units 300, as well as other associated items.

It is understood that any aspect and/or element of any embodiment of the system 10 described herein or otherwise may be combined in any way with any other aspect and/or element of any other embodiment to form additional embodiments of the system 10 all of which are within the scope of the system 10.

Where a process is described herein, those of ordinary skill in the art will appreciate that the process may operate without any user intervention. In another embodiment, the process includes some human intervention (e.g., a step is performed by or with the assistance of a human).

As used herein, including in the claims, the phrase “at least some” means “one or more,” and includes the case of only one. Thus, e.g., the phrase “at least some ABCs” means “one or more ABCs”, and includes the case of only one ABC.

As used herein, including in the claims, term “at least one” should be understood as meaning “one or more”, and therefore includes both embodiments that include one or multiple components. Furthermore, dependent claims that refer to independent claims that describe features with “at least one” have the same meaning, both when the feature is referred to as “the” and “the at least one”.

As used in this description, the term “portion” means some or all. So, for example, “A portion of X” may include some of “X” or all of “X”. In the context of a conversation, the term “portion” means some or all of the conversation.

As used herein, including in the claims, the phrase “using” means “using at least,” and is not exclusive. Thus, e.g., the phrase “using X” means “using at least X.” Unless specifically stated by use of the word “only”, the phrase “using X” does not mean “using only X.”

As used herein, including in the claims, the phrase “based on” means “based in part on” or “based, at least in part, on,” and is not exclusive. Thus, e.g., the phrase “based on factor X” means “based in part on factor X” or “based, at least in part, on factor X.” Unless specifically stated by use of the word “only”, the phrase “based on X” does not mean “based only on X.”

In general, as used herein, including in the claims, unless the word “only” is specifically used in a phrase, it should not be read into that phrase.

As used herein, including in the claims, the phrase “distinct” means “at least partially distinct.” Unless specifically stated, distinct does not mean fully distinct. Thus, e.g., the phrase, “X is distinct from Y” means that “X is at least partially distinct from Y,” and does not mean that “X is fully distinct from Y.” Thus, as used herein, including in the claims, the phrase “X is distinct from Y” means that X differs from Y in at least some way.

It should be appreciated that the words “first,” “second,” and so on, in the description and claims, are used to distinguish or identify, and not to show a serial or numerical limitation. Similarly, letter labels (e.g., “(A)”, “(B)”, “(C)”, and so on, or “(a)”, “(b)”, and so on) and/or numbers (e.g., “(i)”, “(ii)”, and so on) are used to assist in readability and to help distinguish and/or identify, and are not intended to be otherwise limiting or to impose or imply any serial or numerical limitations or orderings. Similarly, words such as “particular,” “specific,” “certain,” and “given,” in the description and claims, if used, are to distinguish or identify, and are not intended to be otherwise limiting.

As used herein, including in the claims, the terms “multiple” and “plurality” mean “two or more,” and include the case of “two.” Thus, e.g., the phrase “multiple ABCs,” means “two or more ABCs,” and includes “two ABCs.” Similarly, e.g., the phrase “multiple PQRs,” means “two or more PQRs,” and includes “two PQRs.”

The present invention also covers the exact terms, features, values and ranges, etc. in case these terms, features, values and ranges etc. are used in conjunction with terms such as about, around, generally, substantially, essentially, at least etc. (i.e., “about 3” or “approximately 3” shall also cover exactly 3 or “substantially constant” shall also cover exactly constant).

As used herein, including in the claims, singular forms of terms are to be construed as also including the plural form and vice versa, unless the context indicates otherwise. Thus, it should be noted that as used herein, the singular forms “a,” “an,” and “the” include plural references unless the context clearly dictates otherwise.

Throughout the description and claims, the terms “comprise”, “including”, “having”, and “contain” and their variations should be understood as meaning “including but not limited to”, and are not intended to exclude other components unless specifically so stated.

It will be appreciated that variations to the embodiments of the invention can be made while still falling within the scope of the invention. Alternative features serving the same, equivalent or similar purpose can replace features disclosed in the specification, unless stated otherwise. Thus, unless stated otherwise, each feature disclosed represents one example of a generic series of equivalent or similar features.

The present invention also covers the exact terms, features, values and ranges, etc. in case these terms, features, values and ranges etc. are used in conjunction with terms such as about, around, generally, substantially, essentially, at least etc. (i.e., “about 3” shall also cover exactly 3 or “substantially constant” shall also cover exactly constant).

Use of exemplary language, such as “for instance”, “such as”, “for example” (“e.g.,”) and the like, is merely intended to better illustrate the invention and does not indicate a limitation on the scope of the invention unless specifically so claimed.

While the invention has been described in connection with what is presently considered to be the most practical and preferred embodiments, it is to be understood that the invention is not to be limited to the disclosed embodiment, but on the contrary, is intended to cover various modifications and equivalent arrangements included within the spirit and scope of the appended claims.

1. A food container sanitizing system comprising:
an ultraviolet germicidal irradiation (UVGI) unit including a cleaning chamber, a display, a food container input mechanism, and one or more ultraviolet light irradiators positioned within the cleaning chamber,
a food container adapted to be unfolded into a flat configuration;
wherein the food container input mechanism is adapted to receive the food container and to move it into the cleaning chamber, and the one or more ultraviolet light irradiators are adapted to emit ultraviolet light onto the food container.

2. The food container sanitizing system of claim 1 wherein the food container input mechanism includes a tray.

3. The food container sanitizing system of claim 3 wherein the tray includes an upper recess adapted to receive the food container in a flat configuration.

4. The food container sanitizing system of claim 1 further comprising one or more handheld ultraviolet light irradiators adapted to emit ultraviolet light.

5. The food container sanitizing system of claim 4 wherein the one or more handheld ultraviolet light irradiators each include a motion detector and a ultraviolet light filtering shield.

6. The food container sanitizing system of claim 1 further comprising an air blower in communication to one or more blower vents configured with the cleaning chamber.

7. The food container sanitizing system of claim 1 wherein the UVGI unit includes an inner volume partitioned into a plurality of compartments.

8. The food container sanitizing system of claim 7 further comprising one or more containers of water located in a first compartment of the plurality of compartments holds, and one or more containers of cleaning solution located in a second compartment of the plurality of compartments.

9. The food container sanitizing system of claim 8 further comprising a water jet configured to release water into the cleaning chamber, the water jet in communication with the one or more containers of water.

10. The food container sanitizing system of claim 8 further comprising a water jet configured to release cleaning solution into the cleaning chamber, the water jet in communication with the one or more containers of cleaning solution.

11. The food container sanitizing system of claim 1 further comprising a hand sanitizing unit.

12. The food container sanitizing system of claim 1 further comprising a debris catcher located beneath the cleaning chamber.

13. The food container sanitizing system of claim 12 wherein the cleaning chamber includes a slanted bottom surface adapted to guide debris from the cleaning chamber to the debris catcher.

14. The food container sanitizing system of claim 1 further comprising a camera and/or a motion detector.

15. The food container sanitizing system of claim 1 further comprising a touchscreen display.
